(19) United States
(12) Patent Application Publication
(10) Pub. No.: US 2024/0197755 A1
(43) Pub. Date: Jun. 20, 2024

(54) Pharmaceutical composition for use in the therapeutic treatment of cancer and complications of cancer

(71) Applicant: Guy Faustin MONKAM NITCHEU, LILLE (FR)

(72) Inventor: Guy Faustin MONKAM NITCHEU, LILLE (FR)

(21) Appl. No.: 16/324,881

(22) PCT Filed: Oct. 25, 2017
(86) PCT No.: PCT/IB2017/056612

§ 371 Date: Feb. 11, 2019

(30) Foreign Application Priority Data
Oct. 31, 2016 (FR) 16/70645
Nov. 8, 2016 (FR) 16/70666
Oct. 23, 2017 (FR) 17/71115

(51) Int. Cl. A61K 31/575; A61K 31/015; A61K 31/05; A61K 31/11; A61K 45/06; A61P 35/00

(52) U.S. Cl. CPC A61K 31/575; A61K 31/015; A61K 31/05; A61K 31/11; A61K 45/06; A61P 35/00

(57) ABSTRACT

The invention relates to a pharmaceutical composition, characterised in that it comprises, as an active substance, a combination of beta-elemene, lupeol and a pharmaceutically active agent selected from cinnamaldehyde, 2-hydroxycinnamaldehyde, 2′- benzoyloxycinnamaldehyde, beta-sitosterol, curcumin and the mixtures thereof.

### [TECHNICAL FIELD OF THE INVENTION]

The present invention relates to a pharmaceutical composition that can be used as a medicament, particularly for the therapeutic treatment of cancers, and specifically of hepatocellular carcinoma.

### [PRIOR ART]

Effective palliative treatment for hepatocellular carcinoma (HCC) has long been considered difficult, because this type of tumor tends to be resistant to conventional cytotoxic chemotherapy. Moreover, aggressive chemotherapy with several non- selective cytotoxic molecules often cannot be offered to cirrhotic patients with compromised liver function due to the high risk of systemic side effects.

Improved knowledge of the molecular processes involved in oncogenesis has led to the identification of new targets for the treatment of cancers. New so-called “targeted” therapies are thus currently available in the treatment of HCC and other tumors. They intervene mainly in the transduction of signals (signals that ask the cell to multiply). The so-called tyrosine kinase pathway is the best known to date. This pathway can be blocked by monoclonal antibodies (Mabs) or enzymatic inhibitors (inib). These drugs include VEGF inhibitors (vascular endothelial growth factor; VEGF is secreted by most tumor cells, partly due to lack of local oxygenation, and promotes angiogenesis), EGFR (Epidermal Growth Factor Receptor) inhibitors, inhibitors of several receptors with tyrosine kinase activity that simultaneously target angiogenesis and cell proliferation, kinase inhibitors, IGF-IR inhibitors, mTOR inhibitors, and MEK-ERK signaling pathway inhibitors.

For the most part, however, these targeted therapies yield relatively modest results in terms of efficiency and survival in HCC and especially in advanced HCC. What is more, the effectiveness of targeted therapies for HCC is still limited by resistance phenomena. Some cancer cells possess or acquire the ability to circumvent the mechanisms of action of medicaments, while others are sensitive at first but develop resistance capabilities over the course of treatment.

It should also be noted that significant side effects associated with these targeted therapies can cause discomfort, thereby denying patients a better quality of life: myelosuppression (blood tests are performed regularly to check red blood cells, white blood cells and platelets), alopecia (which can be psychologically difficult for patients to deal with because it is a concrete and visible sign of the disease), skin disorders and hand-foot syndrome, diarrhea, increased blood pressure, proteinuria, hyperglycemia, hypercholesterolemia, allergic reactions, cytokine release syndrome, tumor lysis syndrome that can self-sustain the tumor, etc.

In addition, several observations point to the existence of a close link between chronic inflammation and the risk of developing cancer. One of the best examples of this correlation is undoubtedly the dramatic (20-fold) increase in the risk of colon cancer in people with inflammatory bowel disease, particularly ulcerative colitis. This also applies to stomach cancer, which develops after an inflammation caused by the presence of a bacterium, Helicobacter pylori. Prostate cancer is caused by inflammation of the prostate (prostatitis). In CHC, the hypothesis is that chronic inflammation increases DNA mutations and that increased proliferation of hepatocytes in regeneration nodules increases the likelihood of attachment of oncogenic mutations. Furthermore, pre- and post-infusion medication that combines a corticosteroid and an antihistamine is strongly recommended in order to reduce the risk of allergic reactions associated with certain targeted therapies.

While biomarkers have been validated for breast cancer (number of copies of HER), lung cancer (EGFR mutations), or colon cancer (Kras mutations), there are currently no validated prognostic biomarkers for HCC under targeted therapy.

A number of studies have shown that reptin and/or pontine are overexpressed during carcinogenesis; their nucleocytoplasmic localization varied depending on the type of cancer and was not necessarily a factor of poor prognosis. On the other hand, the overexpression of reptin and pontine is a factor of poor prognosis in HCC, and a high level of pontine mRNA is correlated with a poor prognosis [(Haurie et al., 2009). Hepatology. 2009 December; 50(6):1871-83. doi: 10.1002/hep.23215].

In addition, it is known that reptin is overexpressed in prostate cancer, hepatocellular carcinoma (HCC), gastric cancer, kidney cancer, and breast cancer.

It is also known that pontine is overexpressed in hepatocellular carcinoma, lung cancer, and colorectal cancer.

The dysfunction of reptin and/or pontine has also been demonstrated in other cancers such as chronic leukemias, mesotheliomas, and multiple myelomas, high-grade lymphomas, Burkitt lymphomas, brain tumors such as gliomas, and bladder tumors.

Survivin is also known as a therapeutic target in the case of cancer. The majority of solid tumors (cancer of the breast, prostate, lung, kidney, etc.) or hematopoietic tumors (multiple myeloma, leukemia, etc.) express it in an aberrant manner. It is also known that cancerous breast, lung, and kidney tumor cells overexpress survivin.

Moreover, it is known that beta-elemene can be used as an anticancer drug. What is more, it has a broad antineoplastic spectrum, including tumors that are resistant to the anticancer drugs that are conventionally used. It is also known to be non-cytotoxic and well tolerated by patients. It is able to pass through the blood-brain barrier and has immunostimulatory properties. Its anti-inflammatory activity is also known. It is also known that beta-elemene inhibits survivin. It is also known to reduce or even suppress the resistance of cancer cells to anticancer drugs.

It is also known that beta-elemene also reduces the resistance of cancer cells to certain drugs. For instance, it has a substantial inhibitory effect on MDR1, MRP, and GST-π. Chen et al., (2006) (Journal of Medicinal Plants Research Vol. 6 (46), pp. 5720-5729, 3 December 2012) have shown that 13-elemene inevitably increases the intracellular accumulation of AMD in U251/AMD cells (cells that have developed a resistance to AMD) of the human glioblastoma and reduces the IC50 of U251/AMD cells from 0.915 to 0.051 mg/l.

In addition, it is also known that lupeol (also known as Fagarasterol or Clerodol), is a pharmacologically active compound with anti-inflammatory, anticancer properties, particularly due to its antiproliferative activity, its regulation of the cell cycle, of apoptosis, and of angiogenesis, and its effect on the epithelial-mesenchymal transition. It also stimulates the immune system of cancer patients. It should be noted that, at the effective therapeutic dose, lupeol exhibits no toxicity to normal cells and tissues.

In the case of CHC, lupeol is known to inhibit the Brain-Derived Neurotrophic Factor (BDNF). Lupeol inhibits cell proliferation of HCCLM3 cells of HCC as a function of concentration and time through activation of Caspase-3 and cleavage of PARP [poly(ADP-ribose)polymerase]. However, Zhang L. et al. (European Journal of Pharmacology, volume 762, 5 Sep. 2015, pages 55-62) also found that the lupeol-induced death of these cells was associated with a marked decrease in the expression of the BDNF protein and ser-9-phosphorylation of GSK-313 (Glycogen Synthase Kinase 3 Beta), with concomitant suppression of the expression of Akt1, PBK (phosphatidylinositol 3-kinase), 13-catenin, c-Myc, and mRNA of cyclin D1. The inhibition of the overexpression of BDNF is therefore a result of the decrease in the expression of the Akt and PI3k proteins as well as the reactivation of the GSK-313 function.

What is more, oral administration of lupeol at a dose of 50 mg/kg for 18 consecutive days produced neither mortality nor systemic toxicity in rats.

It is also known that the cinnamaldehydes—particularly cinnamaldehyde (CA), 2-hydroxycinnamaldehyde (HCA), and 2-benzoyloxycinnamaldehyde (BCA), a semi-synthetic derivative of HCA—each have anti-inflammatory, antiproliferative, antiangiogenic, antimetastatic activity due to the inhibition of the TEM (epithelial-mesenchymal transition) and proapoptotic activity on many human cancer cells such as melanoma, breast cancer, lung cancer, ovarian cancer, colon cancer, prostate cancer, myeloma, and leukemia.

It is also known that HCA has an effect on HCC. Thus, Moon E.Y. (Eun-Yi Moon et al., (2005) European Journal of Pharmacology 530 (2006) 270-275) investigated the inhibitory effect of HCA on farnesyltransferase. In a model mouse that developed hepatocellular carcinoma following a transgenic H-rasl 2V mutation and under the control of a specific promoter such as albumin, it was found that administration of HCA/BCA for 10 weeks delayed the development of liver cancer compared to the control group. HCA/BCA significantly reduces the frequency and extent of liver damage. HCA/BCA increases the number of splenocytes and infiltration of lymphocytes in the liver. These data suggest that delayed onset of liver cancer may be caused by an immunostimulatory effect of HCA/BCA on T cells.

Moreover, beta-sitosterol is known to possess anti-inflammatory, antipyretic, antineoplastic, and immunomodulatory properties.

### [Technical Problem to be Solved]

It is an object of the present invention to provide a novel pharmaceutical composition that can be used as a medicament and more particularly in the treatment of cancer.

Another object of the invention is to provide a novel pharmaceutical composition that can be used as a medicament and more particularly in the treatment of cancer that overcomes all or some of the disadvantages associated with the compositions of the aforementioned prior art.

Another object of the invention is to provide a pharmaceutical composition that is particularly advantageous in the treatment of HCC.

Another object of the present invention is to provide a pharmaceutical composition that can be used as a medicament, particularly for the therapeutic treatment of HCC, of hepatocellular insufficiency, and particularly of cirrhosis of the liver.

Another object of the present invention is to provide a pharmaceutical composition for use in the treatment of breast cancer and/or prostate cancer.

Another object of the present invention is to provide a pharmaceutical composition, particularly as mentioned above, that has reduced toxicity and/or is well tolerated by patients.

Another object of the present invention is to provide a pharmaceutical composition that makes it possible to reduce or inhibit drug resistance and, in particular, drug resistance to anticancer agents.

Another object of the present invention is to provide a pharmaceutical composition that acts specifically on cancer cells that overexpress at least one protein selected from the group consisting of reptin, pontine, and survivin and, in particular, that acts on the cancer cells that overexpress reptin and pontine and possibly survivin.

Another object of the present invention is to provide a pharmaceutical composition that inhibits the role of stromal cells in the overexpression by cancer cells of at least one protein selected from the group consisting of reptin, pontine, and survivin.

Another object of the present invention is to provide a pharmaceutical composition that makes it possible to inhibit the overexpression of CXCR4 receptors by cancer cells and to block the formation of new vascularization, thus reducing the blood supply to growing tumors.

Another object of the present invention is to provide a pharmaceutical composition that makes it possible to inhibit the formation of tumor stroma and the metastatic process, thereby reducing the risk of tumor recurrence.

Another object of the present invention is to provide a pharmaceutical composition that makes it possible to inhibit inflammation, alteration of the intestinal mucosa, bacterial, viral, or fungal translocation from the intestinal lumen into the bloodstream, and systemic immune hyperactivation in order to induce a vigorous immune response, particularly in the lymphoid tissue associated with the digestive tract.

### [BRIEF DESCRIPTION OF THE INVENTION]

In order to solve at least one of the aforementioned technical problems, the present invention provides a pharmaceutical composition that typically comprises, as active substance, a combination of beta-elemene, lupeol, and a pharmaceutically active agent selected from among cinnamaldehyde, 2-hydroxycinnamaldehyde, 2′-benzoyloxycinnalmaldehyde, beta-sitosterol, curcumin, and mixtures thereof.

In fact, the applicant has found that such a pharmaceutical composition is active in the treatment of cancer, particularly in the case of HCC, breast cancer, and prostate cancer.

The applicant has also demonstrated a synergistic effect of at least two of the constituents that provides reinforced action of the composition of the invention on at least one mechanism involved in the cancer phenomenon, namely a mechanism selected from among the formation of the tumor stroma, cell growth, apoptosis, angiogenesis, metastatic process, activation of cell signaling pathways involved in inflammation, lipid and carbohydrate metabolism, and bacterial, viral, and fungal infection.

The applicant has also demonstrated that the composition according to the invention has an effect on cells that overexpress reptin and/or pontin, which is the case of cancer cells of the majority of cancers, including HCC and breast and prostate cancer.

The Applicant has also demonstrated that the composition according to the invention inhibits certain axes, particularly CXCR4/CXCL12, that play a fundamental role in proliferation, tumor growth, metastasis, and the formation of an immunosuppressive microenvironment.

### [DETAILED DESCRIPTION]

The pharmaceutical composition according to the invention can be used as a medicament, particularly for use in the therapeutic treatment of cancer. According to a particular embodiment of the present invention, the composition of the invention can further comprise a mixture of beta-sitosterol and cinnamaldehyde or a mixture of beta-sitosterol and 2-hydroxycinnamaldehyde or a mixture of beta-sitosterol and 2′-benzoyloxycinnamaldehyde or a mixture of cinnamaldehydes, particularly cinnamaldehyde with one or more of its synthetic derivatives and/or metabolites.

Preferably, it does not include a mixture of 2-hydroxycinnamaldehyde, 2′-benzoyloxycinnalmaldehyde, and beta-sitosterol.

By way of example, it can comprise, as a percentage by weight relative to the total weight of the active substances, a percentage by weight of lupeol that is substantially equal to or greater than 15% and substantially equal to or less than 55%, and particularly substantially equal to or greater than 30% and substantially equal to or less than 50%; a percentage by weight of beta-elemene that is substantially equal to or greater than 10% and substantially equal to or less than 55%, and particularly substantially equal to or greater than 20% and substantially equal to or less than 40%; a percentage by weight of cinnamaldehyde that is substantially equal to or greater than 10% and substantially equal to or less than 45%, and particularly substantially equal to or greater than 20% and substantially equal to or less than 40%; a percentage by weight of 2-hydroxycinnamaldehyde that is substantially equal to or greater than 10% and substantially equal to or less than 45%, and particularly substantially equal to or greater than 20% and substantially equal to or less than 40%; a percentage by weight of 2′-benzoyloxycinnalmaldehyde that is substantially equal to or greater than 10% and substantially equal to or less than 45%, and particularly substantially equal to or greater than 20% and substantially equal to or less than 40%; a percentage by weight of beta-sitosterol, when the composition contains this ingredient, that is substantially equal to or greater than 10% and substantially equal to or less than 45%, and particularly substantially equal to or greater than 20% and substantially equal to or less than 40%.

When the composition comprises cinnamaldehyde and one of its derivatives, their percentage by weight relative to the total weight of the active substances is particularly equal to and particularly substantially equal to 20%.

The composition according to the invention further comprises at least one pharmaceutically acceptable excipient. This excipient can be a solid or liquid. It can be selected, for example, from among purified water, ethyl alcohol, propylene glycol, glycerin, vegetable oils, animal oils, hydrocarbons, silicones, sugars such as glucose or levulose, wheat starch, corn starch, potato starch, xanthan gum, gum arabic, tragacanth, karaya gum, guar gum or “guaranates,” pectins, alginates, carrageenates, agar or agar-agar, gelatin, cellulose and derivatives thereof.

The composition of the invention can be administered by any suitable route, for example by oral, rectal, local (topical, for example), intraperitoneal, systemic, intravenous, intramuscular, subcutaneous, or mucosal route, particularly sublingually or using a patch, or encapsulated in, or immobilized on, liposomes, microparticles, microcapsules, or combined with nanoparticles and the like. Some noteworthy, non-limiting examples of excipients that are suitable for oral administration include talc, lactose, starch and derivatives thereof, cellulose and derivatives thereof, polyethylene glycols, acrylic acid polymers, gelatin, magnesium stearate, animal, vegetable, or synthetic fats, paraffin derivatives, glycols, stabilizers, preservatives, antioxidants, wetting agents, anti-caking agents, dispersants, emulsifiers, taste modifiers, penetration agents, and solubilizers. The techniques of the formulation and administration of drugs and pharmaceutical compositions are well known in the relevant art; in particular, a person skilled in the art can refer in particular to the latest edition of the book Remington's Pharmaceutical Sciences.

According to the invention, the composition can be advantageously administered orally by intravenous injection.

Advantageously, the composition according to the invention is suitable for being administered orally or intravenously at a dosage substantially equal to or greater than 40 mg/kg/24 h and substantially equal to or less than 200 mg/kg/24 h in one or more doses to a mammal with such a need.

By way of example, the composition of the invention can be used in the therapeutic treatment of a cancer selected from among hepatocellular carcinoma, colon cancer, rectal cancer, stomach cancer, cancer of the mouth, particularly tongue cancer, prostate cancer, metastatic prostate cancer, kidney cancer, breast cancer, chemoresistant breast cancer, bladder cancer, leukemia in chronic or acute form, multiple myeloma, lymphoma, brain tumors, lung cancer, particularly lung adenocarcinoma, uterine cancer, ovarian cancer, bone tumors, pancreatic cancer, gall bladder cancer, and liver cancer.

The composition according to the invention can be advantageously used in patients with a chronic inflammatory disease, particularly in inflammatory bowel diseases, more particularly ulcerative colitis; in patients carrying a pathogenic agent capable of causing inflammation, particularly Helicobacter pylori; in patients with diabetes, dyslipidemia, or osteoarticular diseases; in patients with hepatocellular insufficiency; in patients with bacterial, fungal, or viral infections, particularly in patients with the hepatitis B virus and/or hepatitis C virus and/or the human immunodeficiency virus (HIV).

In the case of HCC, the applicant has demonstrated that the composition according to the invention yielded good results at least in vitro and exhibited no toxicity for patients' liver cells.

The mode of action of the composition of the invention is not fully understood. It is more than likely that it acts simultaneously on different mechanisms of cancer. Thus, the composition of the invention can be used in the therapeutic treatment of cancer as an agent for blocking the recruitment of bone marrow stem cells to the tumor microenvironment and/or as an inhibitor of tumor stroma formation and/or as an inhibitory agent against the overexpression of at least one protein selected from the group consisting of reptin, pontin, and survivin, preferably reptin and pontin, and/or as an anti-inflammatory agent and/or as an agent that causes apoptosis of cancer cells and/or as an agent for inhibiting angiogenesis and/or as an antimetastatic agent.

This mechanism would consist in the destructuring and restructuring of the lipid composition of cell membranes, thereby inhibiting the cellular signaling pathways involved in metabolic diseases, the secretion of inflammatory cytokines, chemokines, cell proliferation, angiogenesis, metastasis, and in bacterial, viral, or fungal infection.

The present invention also relates to a pharmaceutical preparation that comprises the composition according to the invention, and, in addition, as a mixture or packaged separately, at least one anti-cancer agent for use in the therapeutic treatment of cancer simultaneously, sequentially, or in time intervals.

By way of example, the anticancer agent can be selected from among VEGF inhibitors, EGFR receptor inhibitors, inhibitors of several receptors with tyrosine kinase activity that simultaneously target angiogenesis and cell proliferation, kinase inhibitors, IGF-IR receptor inhibitors, mTOR inhibitors, MEK-ERK pathway inhibitors, paclitaxel, As4S4, tamoxifen, curcumin, vincristine, adriamycin, aclarubicin, oxaliplatin, calcium folinate, 5-fluorouracil, capecitabine, cisplatin, tetramethylpyrazine, methotrexate, daunorubicin, certain genetically modified viruses that preferably target cancer cells, and mixtures thereof, particularly mixtures of two of these anti-cancer agents, radioactive agents that can be used in brachytherapy and/or the injectable or ingestible radioactive metabolites.

The present invention also relates to a pharmaceutical preparation that comprises, in combination, beta-elemene, lupeol, and/or beta-sitosterol, cinnamaldehyde and/or 2-hydroxycinnamaldehyde and/or 2′-benzoyloxycinnamaldehyde, and optionally curcumin.

The present invention also relates to a dietary supplement that comprises, in combination, beta-elemene, lupeol, and/or beta-sitosterol and a pharmaceutically active agent selected from among cinnamaldehyde, 2-hydroxycinnamaldehyde, 2′-benzoyloxycinnalmaldehyde, and mixtures thereof, and optionally curcumin.

### [Definitions]

The term “therapeutic treatment” refers to curative treatment and prophylactic treatment; within the meaning of the present invention, a therapeutic treatment makes it possible to at least partially restore, at least partially correct, or at least partially modify physiological functions by exerting a pharmacological, immunological, or metabolic action.

The term “patient” refers to an animal or human mammal. The composition according to the invention can also be used in veterinary medicine.

For the purposes of the present invention, an “anti-cancer agent” is an element which, at least in vitro, exhibits an action against cancer cells, irrespective of its mechanism of action. For the purposes of the present invention, the term “action” is understood to mean the at least partial destruction or modification of cancer cells which, in particular, makes it possible to limit the proliferation and/or propagation of cancer cells.

The term “patients with diabetes” refers to patients with type 1 diabetes, patients with type 2 diabetes, patients with gestational diabetes, patients with diabetes insipidus, and patients with renal diabetes.

The term “dyslipidemia” refers to hyperlipidemia and hypolipidemia determined according to the current criteria.

The term “patients with osteoarticular diseases” refers to patients who have at least two signs selected from among inflammatory signs, fistulas, and proven bacterial presence detected by draining or blood culture.

The term “patients with hepatocellular insufficiency” refers to patients with hepatitis, regardless of its cause (viral, toxic, drug, or ischemic) and patients with cirrhosis of the liver.

The term “viral infection” refers to a biological entity, whether it is the hepatitis B virus (HBV), the hepatitis C virus (HCV), the human immunodeficiency virus (HIV), the herpes virus (HSV, herpes simplex virus), or cytomegalovirus (CMV), that requires a host, usually a cell, the constituents of which it uses to multiply.

For the purposes of the present invention, a “dietary supplement” is a foodstuff whose purpose it is to supplement the normal diet and which constitutes a concentrated source of nutrients or other substances having a nutritional or physiological effect alone or in combination.

With regard to the cited anti-cancer agents, the terms used, unless otherwise indicated, include the constituent isomers, conformational stereoisomers, enantiomers, and diastereomers of the chemical compound under consideration.

As regards cinnamaldehyde in the composition according to the invention, the term, unless otherwise indicated, includes cinnamaldehyde derivatives, formation dimers—in this case HCA-HCA, BCA-BCA, CA-CA.

### [EXAMPLES]

The percentage of the compositions below is a percentage by weight relative to the total weight of the active substances.

Composition 1a: beta-elemene (30%), lupeol (30%), and 2-hydroxycinnamaldehyde (40%).

Composition 1b: beta-elemene (30%), lupeol (30%), and 2′-benzoyloxycinnamaldehyde (40%).

Composition 2: beta-elemene (30%), lupeol (30%), 2-hydroxycinnamaldehyde (20%), and 2′-benzoyloxycinnamaldehyde (20%).

Composition 3a: beta-elemene (50%), lupeol (30%), and 2-hydroxycinnamaldehyde (20%).

Composition 3b: beta-elemene (50%), lupeol (30%), and 2′-benzoyloxycinnamaldehyde (20%).

Composition 4a: beta-elemene (15%), lupeol (50%), beta-sitosterol (25%), and 2-hydroxycinnamaldehyde (10%).

Composition 4b: beta-elemene (15%), lupeol (50%), beta-sitosterol (25%), and 2′-benzoyloxycinnamaldehyde (10%).

Composition 5a: beta-elemene (20%), lupeol (20%), beta-sitosterol (40%), and 2-hydroxycinnamaldehyde (20%).

Composition 5b: beta-elemene (20%), lupeol (20%), beta-sitosterol (40%), and 2′-benzoyloxycinnamaldehyde (20%).

Composition 6a: beta-elemene (25%), lupeol (35%, beta-sitosterol (15%), and 2-hydroxycinnamaldehyde (25%).

Composition 6b: beta-elemene (25%), lupeol (35%), beta-sitosterol (15%), and 2′-benzoyloxycinnamaldehyde (25%).

Composition 7a: beta-elemene (40%), lupeol (20%), beta-sitosterol (20%), and 2-hydroxycinnamaldehyde (20%).

Composition 7b: beta-elemene (40%), lupeol (20%), beta-sitosterol (20%), and 2′-benzoyloxycinnamaldehyde (20%).

### [Experimental Results]

Different human Hep3B (hepatocellular carcinoma), MCF-7 (breast cancer), DU-145 (prostate cancer) cell lines were studied. They were selected on the basis of their ability to overexpress at least one protein from among reptin, pontin, and survivin. The stromal cells were also studied in order to determine the impact of the composition according to the invention in the tumor microenvironment. These cells were maintained in DMEM and supplemented with 10% fetal bovine serum (FBS) and 1% antibiotic-antimycotic solution (MSP) containing penicillin, streptomycin, and amphotericin B under standard growth conditions (5% CO₂, 37° C., humidified atmosphere). The compositions above were dissolved and diluted in DMSO.

The cells above were treated with increasing solutions for 72 h in complete cell media. All treatment and control protocols were prepared as described previously.

In monotherapy, lupeol and 2-hydroxycinnamaldehyde were observed to have a more pronounced antiproliferative effect on the three lines, including Hep3B, DU-145, and MCF-7, compared to β-elemene after 72 hours of exposure.

Solutions combining the molecules have been studied, including lupeol-ß-elemene, 2-hydroxycinnamaldehyde-B-elemene, and 2-hydroxycinnamaldehyde-lupeol. After 72 hours of exposure, an additive and synergistic effect of the higher 2-hydroxycinnamaldehyde-lupeol combination was observed on Hep3B, DU145, and MCF-7 cell lines compared to the other combinations.

The concomitant combination of Lupeol, B-elemene, and 2-hydroxycinnamaldehyde potentiated and synergized the antiproliferative effects of each molecule on the Hep3B and DU145 cell lines in monotherapy. However, the antiproliferative effect of the combination was less substantial on MCF-7 cell lines.

The effect of this combination on the induction of Hep3B cell apoptosis was determined using annexin V/propidium iodide after 48 hours of treatment. Increased staining of annexin-V was observed in Hep3B cells, whereas minimal staining was observed in untreated control cells.

In a co-culture of cancerous and stromal cells, treatment of confluent (50% confluent) cells with any of the prepared solutions resulted in decreased adhesion associated with cancer cell death, inhibition of stromal cell production of soluble factors found in the tumor microenvironment and involved in the metastatic process and in chemoresistance.

These results as a whole suggest that this pharmaceutical composition inhibits tumor stromal formation and hence metastatic dissemination and chemoresistance. It can be rightfully used in the treatment of hepatocellular carcinoma, breast cancer, and prostate cancer—even in advanced stages.

1-10. (canceled).

11. A pharmaceutical composition, characterized in that it comprises, in combination, beta-elemene, lupeol, and/or beta-sitosterol, cinnamaldehyde and/or 2-hydroxycinnamaldehyde and/or 2′-benzoyloxycinnamaldehyde, and optionally curcumin.

12. The pharmaceutical composition as set forth in claim 11, characterized in that it further comprises a mixture of beta-sitosterol and cinnamaldehyde or a mixture of beta-sitosterol and 2-hydroxycinnamaldehyde or a mixture of beta-sitosterol and 2′-benzoyloxycinnamaldehyde or a mixture of cinnamaldehyde with one or more of its synthetic derivatives and/or metabolites.

13. The pharmaceutical composition as set forth in claim 11, characterized in that it comprises, as a percentage by weight relative to the total weight of the active substances, a percentage by weight of lupeol that is substantially equal to or greater than 15% and substantially equal to or less than 55%, and particularly substantially equal to or greater than 30% and substantially equal to or less than 50%; a percentage by weight of beta-elemene that is substantially equal to or greater than 10% and substantially equal to or less than 55%, and particularly substantially equal to or greater than 20% and substantially equal to or less than 40%; a percentage by weight of cinnamaldehyde that is substantially equal to or greater than 10% and substantially equal to or less than 45%, and particularly substantially equal to or greater than 20% and substantially equal to or less than 40%; a percentage by weight of 2-hydroxycinnamaldehyde that is substantially equal to or greater than 10% and substantially equal to or less than 45%, and particularly substantially equal to or greater than 20% and substantially equal to or less than 40%; a percentage by weight of 2′-benzoyloxycinnalmaldehyde that is substantially equal to or greater than 10% and substantially equal to or less than 45%, and particularly substantially equal to or greater than 20% and substantially equal to or less than 40%; a percentage by weight of beta-sitosterol, when the composition contains this ingredient, that is substantially equal to or greater than 10% and substantially equal to or less than 45%, and particularly substantially equal to or greater than 20% and substantially equal to or less than 40%.

14. The pharmaceutical composition as set forth in claim 10, for use in the therapeutic treatment of a cancer selected from among hepatocellular carcinoma, colon cancer, rectal cancer, stomach cancer, cancer of the mouth, particularly tongue cancer, prostate cancer, metastatic prostate cancer, kidney cancer, breast cancer, chemoresistant breast cancer, bladder cancer, leukemia in chronic or acute form, multiple myeloma, lymphoma, brain tumors, lung cancer, particularly lung adenocarcinoma, uterine cancer, ovarian cancer, bone tumors, pancreatic cancer, gall bladder cancer, and liver cancer.

15. The pharmaceutical composition as set forth in claim 11, for use in the therapeutic treatment of cancer in patients with a chronic inflammatory disease, particularly in inflammatory bowel diseases, more particularly ulcerative colitis;
in patients carrying a pathogenic agent capable of causing inflammation, particularly Helicobacter pylori; the hepatitis B virus and/or hepatitis C virus and/or the human immunodeficiency virus (HIV); in patients with diabetes, dyslipidemia, osteoarticular diseases; and patients with hepatocellular insufficiency.

16. The pharmaceutical composition as set forth in claim 11, which destructures and restructures the lipid composition of cell membranes, thereby inhibiting infection, the secretion of inflammatory cytokines, chemokines, alteration of the mucosa, cell proliferation, angiogenesis, metastasis, and induces a vigorous immune response.

17. The pharmaceutical composition as set forth in claim 11, for use in the treatment of cancer as an agent for blocking the recruitment of bone marrow stem cells to the tumor microenvironment and/or as an inhibitor of tumor stroma formation and/or as an inhibitory agent against the overexpression of at least one protein selected from the group consisting of reptin, pontin, and survivin, preferably reptin and pontin.

18. A pharmaceutical preparation, characterized in that it comprises the composition according to the invention, and, in addition, as a mixture or packaged separately, at least one anti-cancer agent for use in the therapeutic treatment of cancer simultaneously, sequentially, or in time intervals.

19. The pharmaceutical preparation as set forth in claim 18, characterized in that the anti-cancer agent is selected from among VEGF inhibitors, EGFR receptor inhibitors, inhibitors of several receptors with tyrosine kinase activity that simultaneously target angiogenesis and cell proliferation, kinase inhibitors, IGF-IR receptor inhibitors, mTOR inhibitors, MEK-ERK pathway inhibitors, paclitaxel, As4S4, tamoxifen, curcumin, vincristine, adriamycin, aclarubicin, oxaliplatin, calcium folinate, 5-fluorouracil, capecitabine, cisplatin, tetramethylpyrazine, methotrexate, daunorubicin, certain genetically modified viruses that preferably target cancer cells, and mixtures thereof, particularly mixtures of two of these anti-cancer agents, radioactive agents that can be used in brachytherapy and/or the injectable or ingestible radioactive metabolites.
